(19) United States
(12) Patent Application Publication
(10) Pub. No.: US 2022/0378468 A1
(43) Pub. Date: Dec. 1, 2022

(54) STEERABLE NEEDLES

(71) Applicant: JANSSEN BIOTECH, INC., Horsham, NJ (US)

(72) Inventors: Rudolph CEDRO, Jr., Stockton, NJ (US); James FLEMING, Bethlehem, PA (US); Anthony R. DIUBALDI, Jackson, NJ (US); Daniel OLSEN, Califon, NJ (US); Jin PARK, Morris Plains, NJ (US)

(21) Appl. No.: 17/770,942

(22) PCT Filed: Oct. 22, 2020
(86) PCT No.: PCT/IB2020/059941

§ 371 Date: Apr. 21, 2022

Related U.S. Application Data
(60) Provisional application No. 62/925,503, filed on Oct. 24, 2019.

(51) Int. Cl. A61B 17/34

(52) U.S. Cl. CPC A61B 17/3421; A61B 2017/00314

(57) ABSTRACT

Various exemplary steerable needles, methods of using steerable needles, and methods of manufacturing steerable needles are provided. In general, a needle configured to be advanced through soft tissue can be configured to be passively steered in a desired direction through the soft tissue. The needle can include a distal tip configured to facilitate the steering. The needle's distal tip can include a beveled or chamfered edge on one side thereof. The distal tip of the needle can be configured to bend relative to a remainder of the needle in a direction opposite to the side of the distal tip that includes the chamfered edge. The soft tissue can provide resistance to the distal tip being advanced therein such that the distal tip automatically bends in a direction away from the chamfered edge. The needle can also include a bendable portion that is proximal to the distal tip.

### FIELD

The present disclosure relates generally to steerable needles.

### BACKGROUND

Accessing an organ or body space of a patient through tissue of the patient may be needed in the course of medical diagnosis or treatment for a variety of different reasons. For example, an interior area of a tumor or lesion may need to be accessed as part of performing a biopsy or delivering a drug to the tumor or lesion. For another example, access into a vessel or other body lumen may be needed to introduce medication therein or to insert a catheter therein, e.g., for measurement of blood pressure. However, it can be difficult for even experienced medical professionals to access organs and body spaces through tissue quickly and accurately without causing patient discomfort and/or without first experiencing one or more failed attempts to reach the desired area of the organ or body space.

Accordingly, there remains a need for accessing a body area through tissue.

### SUMMARY

In general, steerable needles, methods of using steerable needles, and methods of manufacturing steerable needles are provided.

In one aspect, a medical needle is provided herein. In one embodiment, the medical needle includes a distal portion including a pointed tip and has an asymmetrical shape. The distal portion is configured to be advanced into a soft tissue. The medical needle also includes a proximal portion and an intermediate portion located between the distal and proximal portions. The intermediate portion includes a helical cut therein defining a plurality of male members and a plurality of female members interconnected with the male members. The pointed tip is configured to bend relative to the intermediate and proximal portions, and the intermediate portion is configured to bend relative to the proximal portion.

The medical needle can have any number of variations. For example, the pointed tip can be configured to bend at a hinge. In at least some embodiments, the pointed tip can be configured to automatically bend at the hinge in response to being advanced in a distal direction through soft tissue and/or the hinge can be a uni-directional hinge that allows the pointed tip to bend in only one direction relative to the intermediate and proximal portions. The pointed tip can be defined by a chamfered edge on one side of the distal portion, and the one direction can be in a direction opposite to the side of the distal portion having the chamfered edge thereon.

For another example, the male and female members can each have a trapezoidal shape. For yet another example, the intermediate portion can be cannulated, and the cut can extend fully through a sidewall of the intermediate portion. For still another example, the intermediate portion can not be cannulated, and the cut can extend only partially into an exterior surface of the intermediate portion. For still another example, the distal, proximal, and intermediate portions can be formed from a single tube. For another example, the distal, proximal, and intermediate portions can be cannulated. For still another example, the distal, proximal, and intermediate portions can not be cannulated. For yet another example, the distal portion can not cannulated, and the intermediate portion can be cannulated. For another example, the distal, proximal, and intermediate portions can be metal.

In one embodiment, a medical needle is provided herein that includes a distal portion including a pointed tip and having an asymmetrical shape. The distal portion is configured to be advanced into a soft tissue. The medical needle also includes a proximal portion and an intermediate portion located between the distal and proximal portions. The intermediate portion includes a plurality of discrete segments each including a plurality of male members on one of a proximal end and distal end thereof and a plurality of female members on the other of the proximal end and distal end thereof. Adjacent ones of the segments are connected to one another only by interconnection of the male members and female members. The pointed tip is configured to bend relative to the intermediate and proximal portions, and the intermediate portion is configured to bend along the segments relative to the proximal portion.

The medical needle can have any number of variations. For example, the pointed tip can be configured to bend at a hinge. In at least some embodiments, the pointed tip can be configured to automatically bend at the hinge in response to being advanced in a distal direction through soft tissue and/or the hinge can be a uni-directional hinge that allows the pointed tip to bend in only one direction relative to the intermediate and proximal portions. The pointed tip can be defined by a chamfered edge on one side of the distal portion, and the one direction can be in a direction opposite to the side of the distal portion having the chamfered edge thereon.

For another example, the male and female members can each have a trapezoidal shape. For yet another example, the segments can each have a circular shape with a continuous surface defining an outer perimeter of the circular shape. For still another example, the segments can define a longitudinal member, and the intermediate portion bending along the segments relative to the proximal portion can include the longitudinal member moving from a first configuration, in which longitudinal axes of all of the segments are coaxial with one another, to a second configuration, in which the longitudinal axes of at least some of the segments are not coaxial with one another. For another example, the distal, proximal, and intermediate portions can be cannulated. For still another example, the distal, proximal, and intermediate portions can not be cannulated. For yet another example, the distal portion can not cannulated, and the intermediate portion can be cannulated. For another example, the distal, proximal, and intermediate portions can be metal.

In another aspect, a medical method is provided that in one embodiment includes advancing a needle into soft tissue in a distal direction with a first longitudinal axis defined by a distal portion of the needle aligned with a second longitudinal axis defined by a proximal portion of the needle and aligned with a third longitudinal axis defined by an intermediate portion of the needle. The distal portion of the needle has an asymmetrical shape. The advancement automatically causes the distal portion to bend relative to the proximal and intermediate portions such that the first longitudinal axis becomes misaligned from the second and third longitudinal axes and the advancement thereafter automatically causes the intermediate portion to bend such that the second longitudinal axis becomes misaligned from the third longitudinal axis.

The medical method can have any number of variations. For example, the distal portion can have chamfered edge on one side thereof, and the advancement can automatically cause the distal portion to bend in a direction opposite to the side with the chamfered edge. In at least some embodiments, the medical method can also include, prior to the bending of the distal portion, rotating the needle about a combined longitudinal axis defined by the aligned first, second, and third longitudinal axes to position the chamfered edge in a desired radial position. The needle can not rotate during the bending of the distal portion or during the bending of the intermediate portion.

For another example, the medical method can include, after the bending of the distal and intermediate portions, advancing a surgical instrument through an inner lumen of the needle and out of a distal opening of the distal portion. For yet another example, the needle can have a mandrel positioned in an inner lumen of the needle, and the medical method can include, after the bending of the distal and intermediate portions, removing the mandrel from the inner lumen of the needle, the needle remaining in the soft tissue.

For still another example, the medical method can include delivering a medication to the soft tissue through an inner lumen of the needle and out of a distal opening of the distal portion.

For another example, the needle can be a solid, non-cannulated member. For still another example, the needle can have a flexible sheath positioned thereover, and the medical method can include, after the bending of the distal and intermediate portions, removing the needle from an inner lumen of the flexible sheath, the flexible sheath remaining in the soft tissue. For another example, a pointed tip of the distal portion can bend relative to the proximal and intermediate portions at a hinge of the needle. For yet another example, during the advancement of the needle into soft tissue the intermediate portion can be in a first configuration in which longitudinal axes of all of the segments are coaxial with one another, and the intermediate portion bending can include the longitudinal member moving from the first configuration to a second configuration in which the longitudinal axes of at least some of the segments are not coaxial with one another.

For still another example, the intermediate portion can have a helical cut therein that extends multiple times around a circumference of the intermediate portion.

For another example, the intermediate portion can include a plurality of interconnected discrete segments, each of the plurality of interconnected segments can have a plurality of male members on one of a proximal end and distal end thereof and can have a plurality of female members on the other of the proximal end and distal end thereof, and adjacent ones of the interconnected segments can be connected to one another only by interconnection of the male members and female members. In at least some embodiments, the male and female members can each have a trapezoidal shape.

In yet another example, the distal, proximal, and intermediate portions can be metal.

In another aspect, a method of manufacturing a medical needle is provided that in one embodiment includes cutting a helical shape along a partial longitudinal length of a metal tube to form a bendable portion of a medical needle configured to be advanced into soft tissue. The helical cut defines a plurality of male trapezoidal shapes and a plurality of female trapezoidal shapes that are interconnected with the plurality of male trapezoidal shapes. The medical needle includes, distal to the bendable portion, a metal tip having an asymmetrical shape. The distal tip is configured to lead advancement of the needle into the soft tissue.

The method of manufacturing can have any number of variations. For example, the bendable portion can define a longitudinal member configured to move between a first configuration, in which a longitudinal axis thereof is substantially straight, and a second configuration, in which the longitudinal axis is curved.

For yet another example, the tip can include a chamfered edge, and the tip can include a uni-directional hinge configured to bend a portion of the tip that includes the chamfered edge relative to the bendable portion. For another example, the medical needle can include an elongate metal member proximal to the bendable portion.

For yet another example, the medical needle can be cannulated. For another example, the medical needle can be non-cannulated. For still another example, the metal can be a shape memory material. For yet another example, the metal can be stainless steel.

### BRIEF DESCRIPTION OF DRAWINGS

The present invention is described by way of reference to the accompanying figures which are as follows:

FIG. 1 is a side view of one embodiment of a needle in a first configuration;

FIG. 2 is a perspective view of a distal area of the needle of FIG. 1;

FIG. 3 is another perspective view of a distal area of the needle of FIG. 1;

FIG. 4 is a side, partial view of the needle of FIG. 1 in a second configuration;

FIG. 5 is a side, partial view of the needle of FIG. 1 in a third configuration;

FIG. 6 is a perspective view of a distal area of another embodiment of a needle in a first configuration;

FIG. 7 is a perspective view of a distal area of yet another embodiment of a needle in a first configuration;

FIG. 8 is a side, partial view of the needle of FIG. 1 being rotated and advanced distally relative to soft tissue;

FIG. 9 is a side, partial view of the needle of FIG. 8 being advanced distally relative to soft tissue to move to the second configuration;

FIG. 10 is a side, partial view of the needle of FIG. 9 being advanced distally relative to soft tissue to move to the third configuration;

FIG. 11 is a side, partial view of the needle of FIG. 10 with a flexible tube positioned therein and delivering medication;

FIG. 12 is a side, partial view of the needle of FIG. 8 with a mandrel positioned therein;

and

FIG. 13 is a side, partial view of the needle of FIG. 10 with a flexible sheath positioned thereover.

### DETAILED DESCRIPTION

Certain exemplary embodiments will now be described to provide an overall understanding of the principles of the structure, function, manufacture, and use of the devices, systems, and methods disclosed herein. One or more examples of these embodiments are illustrated in the accompanying drawings. A person skilled in the art will understand that the devices, systems, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. A person skilled in the art will appreciate that a dimension may not be a precise value but nevertheless be considered to be at about that value due to any number of factors such as manufacturing tolerances and sensitivity of measurement equipment. Sizes and shapes of the systems and devices, and the components thereof, can depend at least on the size and shape of components with which the systems and devices will be used.

Various exemplary steerable needles, methods of using steerable needles, and methods of manufacturing steerable needles are provided. In general, a needle configured to be advanced through soft tissue can be configured to be passively steered in a desired direction through the soft tissue. The needle can include a distal tip configured to facilitate the steering. The needle's distal tip can have an asymmetrical shape, which in an exemplary embodiment can include the distal tip having a chamfered edge (also referred to herein as a “beveled edge”) on one side thereof. The distal tip of the needle can be configured to bend relative to a remainder of the needle in a direction opposite to the side of the distal tip that includes the chamfered edge. The soft tissue can provide resistance to the distal tip being advanced therein such that the distal tip automatically bends in a direction away from the chamfered edge. The distal tip can thus be configured to naturally guide the needle in a desired direction in the soft tissue by positioning the chamfered edge opposite to the desired direction of needle bending that would allow for the needle to reach its desired target area.

A portion of the needle that is located proximal to the distal tip can include a bendable portion configured to bend and thus follow the bent distal tip in a curved direction through the soft tissue to facilitate the needle being advanceable through the soft tissue to the target area. In an exemplary embodiment, the bendable portion of the needle includes a helical coil (also referred to herein as a “spiral coil”) that defines a plurality of interconnected protrusions. The protrusions can be connected to one another only by interconnected complementary shapes. The protrusions being connected together using only interconnected ends may reduce cost of the needle and/or may simplify manufacturing of the needle since additional elements such as connector cable(s), connector pin(s), spring element(s), etc. need not be used to connect the protrusions to one another while also allowing the bendable portion to bend. In an exemplary embodiment, the bendable portion is formed of a rigid material such as a biocompatible metal, e.g., stainless steel, a shape memory material (Nitinol, etc.), etc. The bendable portion being formed of a rigid material may provide structural integrity to the needle while still allowing the bendable portion to flex, e.g., by preventing vertical or longitudinal crushing of the bendable portion during the needle's advancement through or retraction from the soft tissue (or elsewhere in a patient's body) and preventing deformation of the bendable portion in response to torque force (e.g., during rotation of the needle along its longitudinal axis).

A needle may be advanced into soft tissue to reach a target area within the soft tissue or a target area located beyond the soft tissue for any of a variety of reasons, e.g., to deliver a medication to the target area, to perform a biopsy at the target area, to capture an image of the target area, etc. It can be difficult, however, to reach the target area because of, e.g., limited available angles of approach to the target area, traditional needles being rigid and unable to bend, etc. The needle being configured to automatically bend in a desired direction may facilitate reaching the target area with the needle. The needle can be introduced into soft tissue at a convenient approach angle and can be positioned within the soft tissue such that the needle's chamfered edge is positioned opposite to the desired angular direction of the needle that will allow the needle to naturally bend toward and be advanced toward the target area.

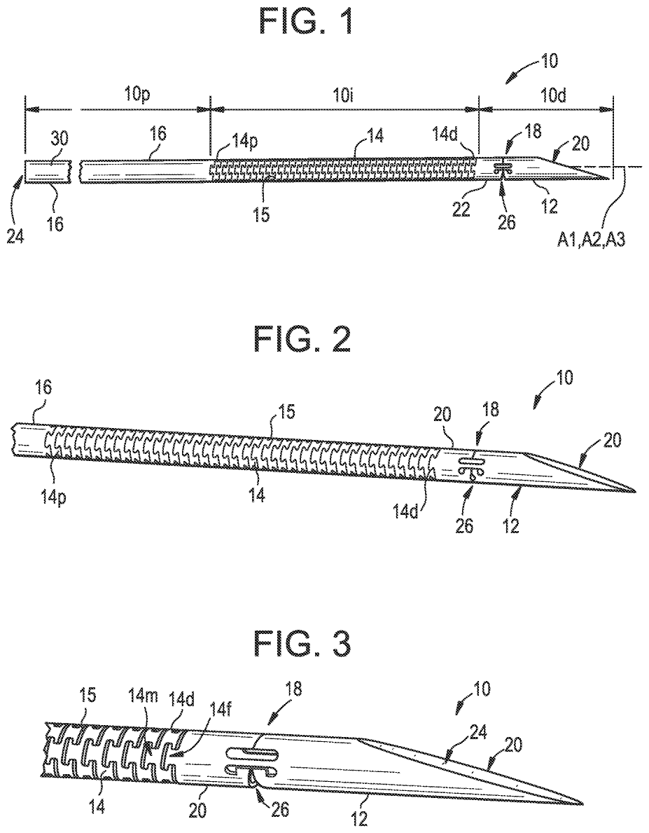

FIGS. 1-5 illustrate one embodiment of a needle 10 configured to be advanced through soft tissue and configured to be passively steered in a desired direction through the soft tissue. The needle 10 includes a distal tip 12, a bendable portion 14 located proximal to the distal tip 12, and an elongate member 16 located proximal to the bendable portion 14. The distal tip 12 is configured to automatically bend relative to the bendable portion 14 and to the elongate member 16 to facilitate the steering of the needle 10. The needle 10 includes a hinge 18 at which the distal tip 12 is configured to bend.

The needle 10 includes a distal portion 10d, a proximal portion 10p, and an intermediate portion 10i defined by the bendable portion 14 that is located between the distal and proximal portions 10d, 10p. The distal tip 12 and the hinge 18 are located in the distal portion 10d of the needle 10. The intermediate portion 10i is located entirely proximal to the distal tip 12 and the hinge 18. The proximal portion 10p includes the elongate member 16.

The distal tip 12 has an asymmetrical shape. The asymmetry is because the distal tip 12 includes a chamfered or beveled edge 20. The chamfered edge 20 is angled and defines a pointed tip of the needle 10. The pointed tip is configured to help the needle 10 penetrate into and through soft tissue when the needle 10 is being advanced in a distal direction into and within soft tissue. The chamfered edge 20 is located on one side of the needle 10, e.g., an upper or top side of the needle 10 as the needle 10 is illustrated in FIG. 1. In other embodiments, the distal tip 12 can have an asymmetrical shape but not includes the chamfered edge 20 but instead have another shape such that a first side, e.g., an upper or top side, of the needle 10 has a different shape than a second, opposite side, e.g., a lower of bottom side, of the needle 10. For example, a Huber-point needle has an asymmetrical shape but does not includes a chamfered edge at its distal tip.

The needle 10 is cannulated in this illustrated embodiment is a tube and has an inner lumen 24 extending therethrough, e.g., through the proximal, intermediate, and distal portions 10p, 10i, 10d. In an exemplary embodiment, as shown in FIG. 1, a sidewall of the tube is solid in the proximal and distal portions 10p, 10d, and the sidewall of the tube in the intermediate portion 10i is not solid has a cut 15 therein that is helical or spiral in shape such that the bendable portion 14 is a helical or spiral coil. The cut 15 extends fully through the sidewall of the tube in this illustrated embodiment, which may provide greater flexibility of the bendable portion 14 as compared to a cut 15 that extends only partially through the sidewall of the tube. The cut 15 can, however, extend only partially through the sidewall of the tube, which may be desirable for needles in certain applications. The cut 15 extends at least 360° around the circumference of the needle 10 and, in an exemplary embodiment extends at least 720° around the circumference of the needle 10. In general, the greater the number of times the cut 15 wraps 360° around the circumference of the needle 10, and hence the greater a longitudinal length of the intermediate portion 10i, the more the needle 10 is configured to bend along the intermediate portion 10i that includes the cut 15.

The cut 15 defines a plurality of protrusions that are connected together so as to interlock. A distal-most one of the protrusions 14d at a distal-most end of the cut 15 is connected to a proximal end of the distal portion 10d. Similarly, a proximal-most one 14p of the protrusions 14 at a proximal-most end of the cut 15 is connected to a distal end of the proximal portion 10p

The cut 15 defines the protrusions as interconnected male and female members that are mated together in dovetail fashion. The protrusions are connected together (and in the case of the distal-most protrusions 14d to the distal portion 10d and of the proximal-most protrusions 14p to the proximal portion 10p) only by being interconnected with the male and female members 14m, 14f at their adjacent proximal and distal ends. As mentioned above, this sole attachment mechanism for the protrusions may reduce cost of the needle 10 and/or may simplify manufacturing of the needle 10.

The cut 15 defines a plurality of female members 14f formed in a distal end surface of the tube and a plurality of male members 14m in a proximal end surface of the tube. The proximal end of the distal portion 10d, e.g., a proximal base member 22 thereof, has a plurality of male members 14m formed in a proximal end surface thereof that are connected to the plurality of female members 14f of the distal-most protrusions 14d. The distal end of the proximal portion 10p, e.g., the elongate member 16 thereof, has a plurality of female members 14f formed in a distal end surface thereof that are connected to the plurality of male members 14m of the proximal-most protrusions 14p. Although in the illustrated embodiment the male members 14m are on proximal end surfaces and the female members 14f are on distal end surfaces, in other embodiments the male members 14m are on distal end surfaces and the female members 14f are on proximal end surfaces.

As best shown in FIG. 3, the male members 14m and the female member 14f do not have straight edges, e.g., edges that are substantially perpendicular to the longitudinal axis A2 of the intermediate portion 10i. Instead, the edges of the male members 14m and the female members 14f are angled non-perpendicularly to the longitudinal axis A2 of the intermediate portion 10i. The angled edges are configured to facilitate secure interconnection of the male and female members 14m, 14f and thus the secure interconnection of adjacent protrusions, the secure interconnection of the distal-most protrusions 14d to the base member 22, and the secure interconnection of the proximal-most protrusions 14p to the elongate member 16.

The male and female members 14m, 14f have complementary shapes such that the male members 14m are configured to lock into the female members 14f. In an exemplary embodiment, as in this illustrated embodiment, the male and female members 14m, 14f have complementary trapezoidal shapes. A trapezoid shape may help ensure that none of the male and female members 14m, 14f become detached from one another (or from the distal portion 10d or the proximal portion 10p) due to the overhanging ends at each side end of the shape.

A number of male members 14m formed in a proximal end surface (and in the distal portion's proximal end) around a single 360° turn of the cut 15 is equal to a number of female members 14f formed in a distal end surface (and in the proximal portion's distal end). Any plural number of male members 14m can be formed in the proximal end surfaces, and any plural number of female members 14f can be formed in the distal end surfaces. In the illustrated embodiment, four male members 14m are formed in the proximal end surfaces, and four female members 14f are formed in the distal end surfaces.

The intermediate portion 10i has a cylindrical shape in this illustrated embodiment. A cylindrical shape may facilitate passage of the needle 10 through surgical cannulas, trocars, scoping devices, etc. and/or may facilitate rotation of the needle 10 about its longitudinal axis. As the needle 10 is cannulated, the cylindrical intermediate portion 10i is thus hollow.

In an exemplary embodiment, the needle 10 is formed of a rigid material such as a biocompatible metal, e.g., stainless steel, a shape memory material (Nitinol, etc.), etc. A rigid material may provide sufficient structural integrity to the needle 10 to allow the needle 10 to be advanced in a distal direction into and within soft tissue without the needle 10 buckling and/or to allow the needle 10 to be rotated into and within soft tissue (similar to a drill bit) without the needle 10 buckling or straying off a substantially straight rotational axis. A material of the distal and proximal portions 10d, 10p is the same material as the intermediate portion 10 in this illustrated embodiment, which may facilitate manufacturing such as by, as in this illustrated embodiment, the cut 15 being formed in a single tube that defines all of the proximal, intermediate, and distal portions 10p, 10i, 10d. In other embodiments, however, the portions 10p, 10d, 10i of the needle 10 can be formed of material different from one or both of the other portions 10p, 10d, 10i.

The male and female members 14m, 14f can be formed in a variety of ways. In an exemplary embodiment, the cut 15 is formed by cutting (e.g., laser cutting, etc.) the male members 14m and the female members 14f in a tube. The male and female members 14m, 14f can be similarly cut in the distal end surface of the proximal portion 10p and the proximal end surface of the distal portion 10d. In another embodiment, the intermediate portion 10i is 3D printed with the male and female members 14m, 14f formed therein as part of the printing process. The male and female members 14m, 14f of the proximal and distal portions 10p, 10d can also be 3D printed, either as part of the same tube including the intermediate portion 10i or as separate tubes that are subsequently connected to the intermediate portion 10i.

In another embodiment, instead of including the spiral cut 15 therein, the intermediate portion 10i can include a plurality of segments that are discrete members connected to one another only by interconnected complementary shapes at their adjacent proximal and distal ends. The needle 10 can include any plural number of segments. In general, the greater the number of segments, and hence the greater a longitudinal length of the intermediate portion 10i, the more the needle 10 is configured to bend along the intermediate portion 10i that includes the segments.

A distal-most one of the segments is connected to a proximal end of the distal portion 10d and to a distal end of a next-most distal one of the segments. Similarly, a proximal-most one of the segments is connected to a distal end of the proximal portion 10p and to a proximal end of a next-most proximal one of the segments. The segments are connected to one another (and in the case of the distal-most segment 14d to the distal portion 10d and of the proximal-most segment 14p to the proximal portion 10p) using interconnected male and female members 14m, 14f that are mated together in dovetail fashion as discussed above with respect to the male and female members 14m, 14f defined by the cut 15.

Each of the segments includes a plurality of female members 14f formed in a distal end surface thereof and a plurality of male members 14m formed in a proximal end surface thereof. The proximal end of the distal portion 10d, e.g., the proximal base member 22 thereof, has a plurality of male members 14m formed in a proximal end surface thereof that are connected to the plurality of female members 14f of the distal-most segment. The distal end of the proximal portion 10p, e.g., the elongate member 16 thereof, has a plurality of female members 14f formed in a distal end surface thereof that are connected to the plurality of male members 14m of the proximal-most segment. Although in the illustrated embodiment the male members 14m are on proximal end surfaces and the female members 14f are on distal end surfaces, in other embodiments the male members 14m are on distal end surfaces and the female members 14f are on proximal end surfaces.

Each of the segments has a circular shape in an exemplary embodiment such that the intermediate portion 10i has a cylindrical shape. Each of the segments is a ring when the needle 10 is cannulated such that each of the segments has a central opening therein and has a continuous surface defining an outer perimeter thereof. The cylindrical intermediate portion 10i is thus hollow. The male and female members 14m, 14f are formed in the continuous outer surface defining the segment outer perimeter. In other embodiments, the needle 10 can be non-cannulated and the segments can be solid members, e.g., discs, without any central opening therein and with the male and female members 14m, 14f formed in the continuous outer surface defining the disc outer perimeter.

In an exemplary embodiment, each of the segments is a rigid member formed of a rigid material such as a biocompatible metal, e.g., stainless steel, a shape memory material (Nitinol, etc.), etc. As mentioned above, rigid material may provide sufficient structural integrity to the needle 10 to allow the needle 10 to be advanced in a distal direction into and within soft tissue without the needle 10 buckling and/or to allow the needle 10 to be rotated into and within soft tissue (similar to a drill bit) without the needle 10 buckling or straying off a substantially straight rotational axis. A material of the distal and proximal portions 10d, 10p can be the same material as the segment material, which may facilitate manufacturing. In other embodiments, however, the segments can be formed of material different from one or both of the proximal and distal portions 10p, 10d.

The male and female members 14m, 14f can be formed in a variety of ways. In an exemplary embodiment, the segments 14 are each formed by cutting (e.g., laser cutting, etc.) the male members 14m and the female members 14f into end surfaces of a ring (or a disc). The male and female members 14m, 14f can be similarly cut in the distal end surface of the proximal portion 10p and the proximal end surface of the distal portion 10d. In another embodiment, the segments 14 are each 3D printed with the male and female members 14m, 14f formed therein as part of the printing process. The male and female members 14m, 14f of the proximal and distal portions 10p, 10d can also be 3D printed.

After the segments have been formed and include their male and female members 14m, 14f (formed in any of a variety of ways, as discussed above), the segments can be connected together to form the intermediate portion 10i. Then, the proximal-most segment of the connected segments can be attached to the distal end of the proximal portion 10p, e.g., the distal end of the elongate member 16, and the distal-most segment of the connected segments can be attached to the proximal end of the distal portion 10d, e.g., the proximal end of the base member 22. The proximal-most segment or the distal-most segment can be connected first to their respective portion 10p, 10d.

Alternatively, instead of the segments being connected together to form the intermediate portion 10i before either of the proximal-most segment 14p and the distal-most segment 14d is connected to their respective portion 10p, 10d, the segments can be connected together in another order. For example, the proximal-most segment 14p can be attached to the distal end of the proximal portion 10p, e.g., the distal end of the elongate member 16, with successive segments being connected to one another in a distal direction finishing with the distal-most segment of the connected segments being attached to the proximal end of the distal portion 10d, e.g., the proximal end of the base member 22. For another example, the distal-most segment can be attached to the proximal end of the distal portion 10d, e.g., the proximal end of the base member 22, with successive segments being connected to one another in a proximal direction finishing with the proximal-most segment of the connected segments being attached to the distal end of the proximal portion 10p, e.g., the distal end of the elongate member 16.

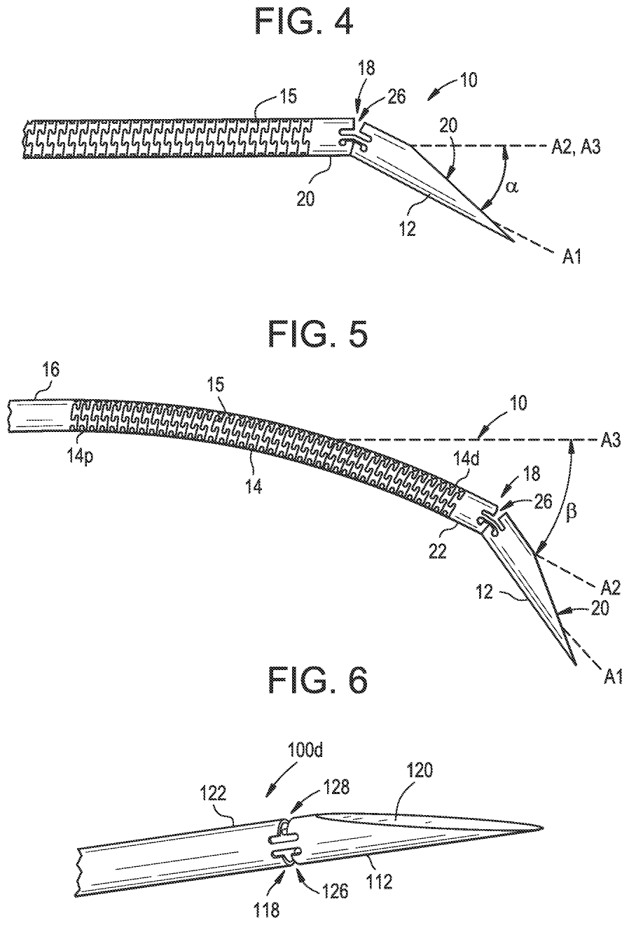

Referring again to the embodiment of FIG. 1, the hinge 18 is located distal to the cut 15 (and in embodiments instead including segments, distal to the segments) and is configured to allow the distal tip 12 to bend to a side opposite to the side of the needle 10 that includes the chamfered edge 20, e.g., down to or the bottom of the needle 10 as the needle 10 is illustrated in FIG. 1. FIGS. 4 and 5 illustrate the distal tip 12 articulated at the hinge 18. The hinge 18 is a living hinge in this illustrated embodiment but can have other configurations.

The hinge 18 in this illustrated embodiment is an integral part of the needle 10, which may facilitate manufacturing of the needle 10 since a discrete hinge need not be attached to the needle 10. The hinge 18 includes a longitudinal extension extending between the proximal base member 22 and the distal tip 12 that includes the chamfered edge 20.

The hinge 18 includes two hinges 18 identical to one another, with the hinges 18 being on opposite sides of the needle 10 as best shown in FIG. 3. The hinges 18 are arranged about 180° apart from one another around the circumference or perimeter of the needle 10. A person skilled in the art will appreciate that the hinges 18 may not be spaced precisely 180° from one another but nevertheless be considered to be about 180° apart from one another due to any number of factors, such as manufacturing tolerances and sensitivity of measurement equipment.

As mentioned above, the needle 10 is cannulated in this illustrated embodiment. The needle 10 being cannulated allows for passage of element(s) through the needle 10. Examples of elements that can be passed through the inner lumen 24 include medication, air, and a surgical instrument (e.g., a biopsy tool, a scoping device with image gathering ability, a guide pin, a mandrel, a guide tube, etc.). Examples of medication that can be delivered to through the inner lumen 24 of the needle 10 include chemo-therapeutic agents, oncologic agents, small molecule agents, large molecule agents, and immunotherapeutic agents. The element(s) can be advanced distally through the inner lumen 24 by being inserted into an opening at the needle's proximal end, e.g., an open proximal end of the elongate element 16, and out an opening at the needle's distal end, e.g., an open distal end of the distal tip 12. In addition to or instead of element(s) being advanced distally through the inner lumen 24, element(s) can be advanced proximally through the inner lumen 24. For example, element(s) advanced distally through the inner lumen 24 but not intended to remain within the patient's body can be retracted and moved proximally in the inner lumen 24 to be removed from the needle 10. For another example, air can be suctioned in a proximal direction through the needle 10 to, e.g., help clear the target area of debris, excess fluid, etc.

In other embodiments, the needle 10 can be non-cannulated. The needle 10 being non-cannulated may increase structural integrity of the needle 10 (as compared to a cannulated needle 10). Thus, if no elements will be passed through the needle 10 during use, the needle 10 being solid may be preferred. For example, as discussed further below, a flexible sheath can be positioned over the needle 10 and used to advance element(s) to a target area instead of element(s) being passed through the needle 10 to the target area.

The needle 10 is configured to move from a first configuration, shown in FIGS. 1-3, to a second configuration, shown in FIG. 4. In the first configuration, the needle 10 is substantially straight. A person skilled in the art will appreciate that the needle 10 may not be precisely straight but nevertheless be considered to be substantially straight due to any number of factors such as manufacturing tolerances, sensitivity of measurement equipment, etc. With the needle 10 in the first configuration, a longitudinal axis A1 of the distal portion 10d, a longitudinal axis A2 of the intermediate portion 10i, and a longitudinal axis A3 of the proximal portion 10p are coaxial with one another.

The longitudinal extension of the hinge 18 that extends between the proximal base member 22 and the distal tip 12 is in a substantially straight configuration when the needle 10 is in the first configuration. Additionally, as shown in FIGS. 1-3, a sloped proximal end surface of the distal tip 12 and a sloped distal end surface of the base member 22 define a cut-out 26 located on one side (e.g., a lower or bottom side as the needle 10 is illustrated in FIG. 1) of the hinge 18 when the needle 10 is in the first configuration, e.g., when the distal tip 12 is not bent. The side of the needle 10 with the cut-out 26 is opposite to the side of the needle 10 that has the chamfered edge 20. The cut-out 26 may facilitate bending of the distal tip 12 as discussed further below. On an opposite side of the hinge 18 (e.g., an upper or top side as the needle 10 is illustrated in FIG. 1) the proximal end surface of the distal tip 12 and the distal end surface of the base member 22 are linear and not sloped and abut one another when the needle 10 is in the first configuration. The abutment of the proximal end surface of the distal tip 12 and the distal end surface of the base member 22 may help ensure that the distal tip 12 is in a substantially straight configuration aligned with the intermediate and proximal portions 10a, 10p.

The first configuration can be the default configuration of the needle 10. For example, in embodiments in which at least the intermediate and distal portion 10i, 10d of the needle 10 are made from a shape memory material, the needle 10 can be configured to naturally move to the first configuration.

In the second configuration, the distal tip 12 is bent at the hinge 18 at an angle α relative to a remainder of the needle 10, e.g., relative to a portion of the needle 10 proximal to the hinge 18 including the base member 22, the intermediate portion 10i, and the proximal portion 10p. The distal tip 12 is configured to bend at the hinge 18 until the sloped proximal end surface of the distal tip 12 and the sloped distal end surface of the base member 22 abut one another. Accordingly, the cut-out 26 no longer appears with the needle 10 in the second configuration. A slope angle of the sloped proximal end surface of the distal tip 12 and the sloped distal end surface of the base member 22 therefore defines the angle α at which the distal tip 12 is configured to bend. In general, the greater the slope angle, the greater the angle α at which the distal tip 12 is configured to bend. Additionally, with the needle 10 in the second configuration, the linear proximal end surface of the distal tip 12 and the linear distal end surface of the base member 22 no longer abut one another. A gap of space 28 thus exists between the linear proximal end surface of the distal tip 12 and the linear distal end surface of the base member 22 with the needle 10 in the second configuration. A size of the gap of space 28 corresponds to a size of the cut-out 26.

With the needle 10 in the second configuration, the longitudinal axis A1 of the distal portion 10d is no longer coaxial with the still-coaxial longitudinal axis A2 of the intermediate portion 10i, and longitudinal axis A3 of the proximal portion 10p. Instead, the distal portion's longitudinal axis A1 is angled at the angle α relative to the intermediate and proximal portions' longitudinal axes A2, A3

The needle 10 is configured to move from the second configuration to a third configuration, shown in FIG. 5. In the third configuration, the distal tip 12 remains bent at the hinge 18 at the angle α. Unlike in the second configuration, in the third configuration the intermediate portion 10i is bent such that the intermediate portion's longitudinal axis A2 is no longer coaxial with the proximal portion's longitudinal axis A3 but is instead at an angle relative thereto. The intermediate portion 10p is configured to bend at the angle β by following the bend of the distal tip 12 as the needle 10 is advanced in a distal direction.

The needle 10 is configured to move from the third configuration back to the second configuration and then from the second configuration back to the first configuration. The needle 10 can be manually urged from the third configuration back to the second configuration and then from the second configuration back to the first configuration. However, in addition to or instead of manual force, the needle 10 being formed of a shape memory material may allow the needle 10 to naturally return to the first configuration from the third configuration.

The needle 10 in this illustrated embodiment includes a single bendable portion 14. In another embodiment, the needle 10 can include a plurality of groups of bendable portions 14 with each of the bendable portions being similar to the single bendable portion 14 in the illustrated embodiment. Adjacent bendable portion 14 can be separated from one another with an elongate member similar to the elongate member 16 and having male and female members 14m, 14f therein to connect to adjacent bendable portions. Including two or more bendable portions 14 with adjacent bendable portions 14 being separated by a non-bendable portion may improve structural integrity of the needle 10 while still providing for needle flexibility by providing non-cut sections of the needle 10 (or, for embodiments including discrete segments instead of the cut 15, by providing a non-segmented section of the needle 10) along the length of the intermediate portion 10i.

FIG. 6 illustrates another embodiment of a distal portion 100d of a needle. Proximal and intermediate portions of the needle of FIG. 6 are omitted for clarity of illustration. The needle of FIG. 6 is generally configured and used similar to the needle 10 of FIGS. 1-5, e.g., is configured to move from a first configuration to a second configuration to a third configuration and back to the first configuration. The distal portion 100d includes a proximal base member 122 and includes a distal tip 112 with a chamfered edge 120. The distal tip 112 is configured to bend at a hinge 118. The hinge 118 includes a longitudinal extension that extends between the proximal base member 122 and the distal tip 112 in a substantially straight configuration when the needle is in its first configuration, similar to that discussed above regarding the distal tip 12 and hinge 18. In the illustrated embodiment of FIG. 6, however, the needle includes a first cut-out 126 on one side of the needle (e.g., a lower or bottom side as the needle is illustrated in FIG. 6) and a second cut-out 128 on an opposite side of the needle (e.g., an upper or top side as the needle is illustrated in FIG. 6). Additionally, a proximal end surface of the distal tip 112 and a distal end surface of the base member 122 that define the first cut-out 126 are linear, e.g., are not sloped. Similarly, a proximal end surface of the distal tip 112 and a distal end surface of the base member 122 that define the second cut-out 128 are linear. When the needle is in the second configuration and in the third configuration, the first cut-out 126 is reduced is size with the proximal end surface of the distal tip 112 and the distal end surface of the base member 122 that define the first cut-out 126 abutting at a distal edge thereof. When the needle is in the second configuration and in the third configuration, the second cut-out 128 is increased in size. The hinge 218 includes two hinges 218 identical to one another, with the hinges 218 being on opposite sides of the needle similar to that discussed above regarding the hinge 18.

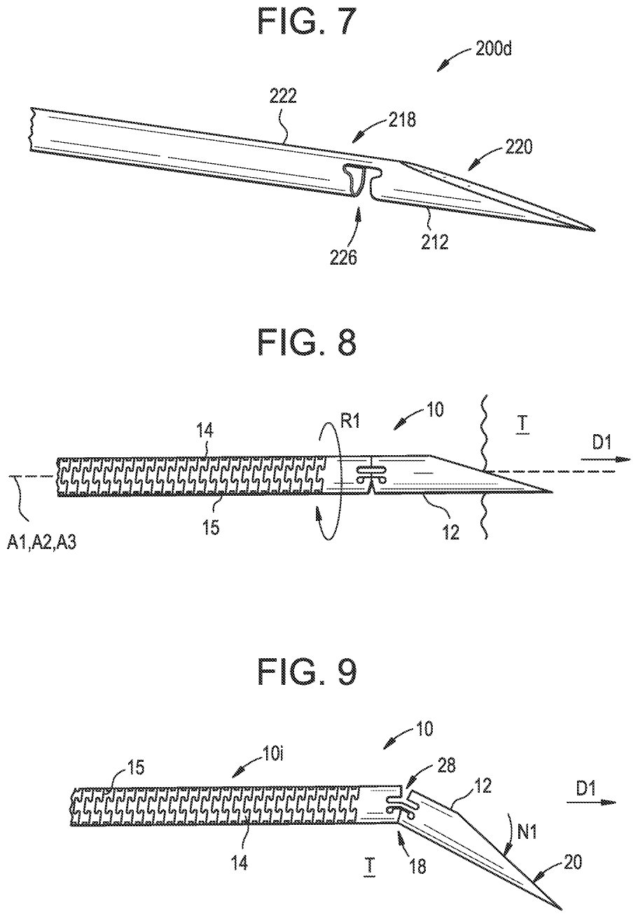

FIG. 7 illustrates another embodiment of a distal portion 200d of a needle. Proximal and intermediate portions of the needle of FIG. 7 are omitted for clarity of illustration. The needle of FIG. 7 is generally configured and used similar to the needle 10 of FIGS. 1-5, e.g., is configured to move from a first configuration to a second configuration to a third configuration and back to the first configuration. The distal portion 200d includes a proximal base member 222 and includes a distal tip 212 with a chamfered edge 220. The distal tip 212 is configured to bend at a hinge 218. The hinge 218 includes a longitudinal extension that extends between the proximal base member 222 and the distal tip 212 in a substantially straight configuration when the needle is in its first configuration, similar to that discussed above regarding the distal tip 12 and hinge 18. The needle includes a cut-out 226 on one side of the needle (e.g., a lower or bottom side as the needle is illustrated in FIG. 7). A proximal end surface of the distal tip 212 and a distal end surface of the base member 222 that define the cut-out 126 are linear, e.g., are not sloped, with rounded extensions at one end thereof. In the illustrated embodiment of FIG. 7, the needle only includes a single hinge 218. The hinge 218 is located on one side of the needle, e.g., the upper or top side as the needle is illustrated in FIG. 7. When the needle is in the second configuration and in the third configuration, the cut-out 226 is reduced is size with the proximal end surface of the distal tip 212 and the distal end surface of the base member 222 that define the cut-out 226 abutting at a distal edge thereof.

FIGS. 8-11 illustrate one embodiment of a method of using a needle as described herein. Although the method is described with respect to the needle 10 illustrated in FIGS. 1-5, other embodiments of needles described herein can be similarly used.

The needle 10 is introduced into a body of a patient, e.g., through an incision, through a trocar, etc., and is positioned relative to a soft tissue T in the patient's body. As shown in FIG. 8, with the needle 10 in the first configuration, the needle 10 is rotated R1 about the common longitudinal axis defined by the coaxial longitudinal axes A1, A2, A3 and advanced in a distal direction D1 in the soft tissue T. Rotating the needle 10 during its distal movement in the soft tissue T prevents the distal tip 12 from naturally bending at the hinge 18. The rotation R1 shown in FIG. 8 is in a counterclockwise direction, but the rotation can instead be clockwise. The distal tip 12 can be located entirely outside the soft tissue T before the rotation R1 begins, or the distal tip 12 can be first at least partially inserted into the soft tissue T. Whether the needle 10 is rotating or not when the needle's pointed tip begins to enter the soft tissue T, the needle's pointed tip T can facilitate the needle's penetration into the soft tissue T.

The rotation R1 of the needle 10 can result in an unpredictable orientation of the distal tip 12, and hence the chamfered edge 20, within the soft tissue T when the rotation R1 stops. Since the position of the chamfered edge 20 defines the direction that the distal tip 12 will bend at the hinge 18, the needle 10 may or may not be in a desired position when the rotation ends R1. The needle 10 may therefore be rotated any number of times until the chamfered edge 20 is in a position to allow the distal tip 12 to bend in a direction toward the target area within (or beyond) the soft tissue T. The proximal portion 10p of the needle 10 can have a marker 30 thereon, as shown in FIG. 1, that is configured to be located outside of the patient's body or in some other location that is visible to a user, e.g., within the patient's body and viewable by eye or via visualization device, when the distal tip 12 is positioned within the soft tissue T. The marker 30 is aligned with and on a same side of the needle 10 as the chamfered edge 20. Thus, the position of the marker 30 will indicate where the chamfered edge 20 is located. The marker 30 is a circular dot on the elongate member 16 in this illustrated embodiment but can have other configurations, e.g., a mark of another shape (e.g., square, triangle, etc.), line(s), number(s), text, cut-out, indentation, etc.

Once the chamfered edge 20 is in a desired position relative to the soft tissue, the needle 10 is advanced in the distal direction D1 without rotation of the needle 10, as shown in FIG. 9, which moves the needle 10 from the first configuration to the second configuration. The soft tissue T provides resistance against the distal tip 12, which forces the distal tip 12 to bend at the hinge 18 in a direction N1, which is a downward direction in the view of FIG. 9 in which the chamfered edge 20 is on an upper side of the needle 10. The distal tip 12 bends until the cut-out 26 disappears and the gap of space 28 appears. The intermediate portion 10i of the needle 10 does not bend during this distal advancement of the needle 10 that moves the needle 10 from the first configuration to the second configuration because of the structural integrity of the intermediate portion 10i (and, if present, a mandrel extending through the intermediate portion 10i, as discussed further below).

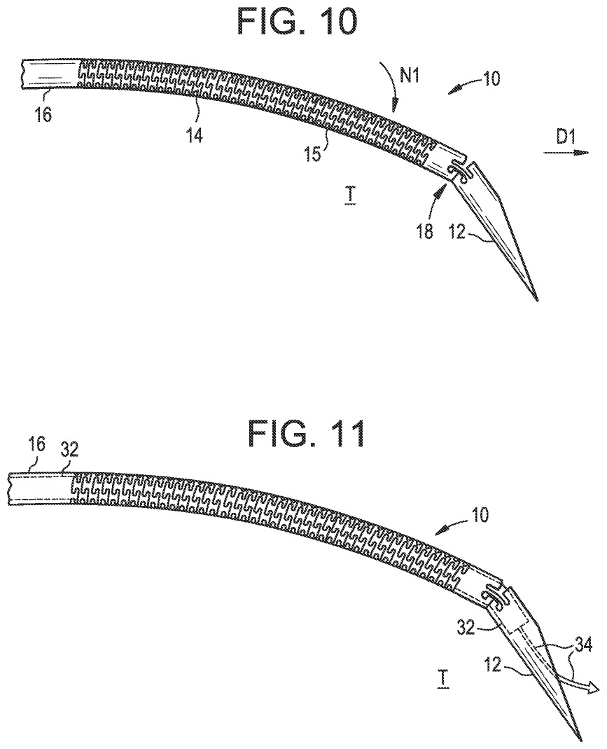

Continued advancement of the needle 10 in the distal direction D1, as shown in FIG. 10, causes the intermediate portion 10i to follow the distal tip's new angular orientation and bend in the direction N1 such that the needle 10 moves from the second configuration to the third configuration. The needle 10 continues to be advanced in the distal direction D1 until the distal tip 12 is positioned as desired relative to the target area, which in this illustrated embodiment is an area within the soft tissue T.

With the distal tip 12 positioned as desired relative to the target area, a flexible elongate tube 32 is advanced distally through the needle's inner lumen 24 until a distal end of the flexible elongate tube 32 is positioned in the distal tip 12 or, if sufficient space exists and/or if the soft tissue is pliant enough, until the distal end of the flexible elongate tube 32 exits the distal opening of the inner lumen 24 at the distal tip 12. Then, as shown in FIG. 11, a medication 34 is passed through the flexible elongate tube 32 to the target area for treatment.

After delivery of the medication 34, the flexible elongate tube 32 is retracted proximally out of the needle 10. The needle 10 is then retracted proximally out of the patient's body, moving from the third configuration to the second configuration and then to the first configuration in the process. Alternatively, the needle 10 and the flexible elongate tube 32 can be retracted proximally out of the patient's body as a unit.

In another embodiment, instead of the flexible elongate tube 32 being advanced through the needle 10 and delivering medication 34, a different surgical instrument can be advanced distally through the needle's inner lumen 24 until a distal end of the surgical instrument is positioned in the distal tip 12 or, if sufficient space exists and/or if the soft tissue is pliant enough, until the distal end of the surgical instrument exits the distal opening of the inner lumen 24 at the distal tip 12. The surgical instrument can then perform action as needed, e.g., being used as a suction device, retrieving a biopsy sample of the soft tissue T, etc. In some embodiments, the medication 34 can be delivered and the surgical instrument can be used before or after the delivery of the medication 34.

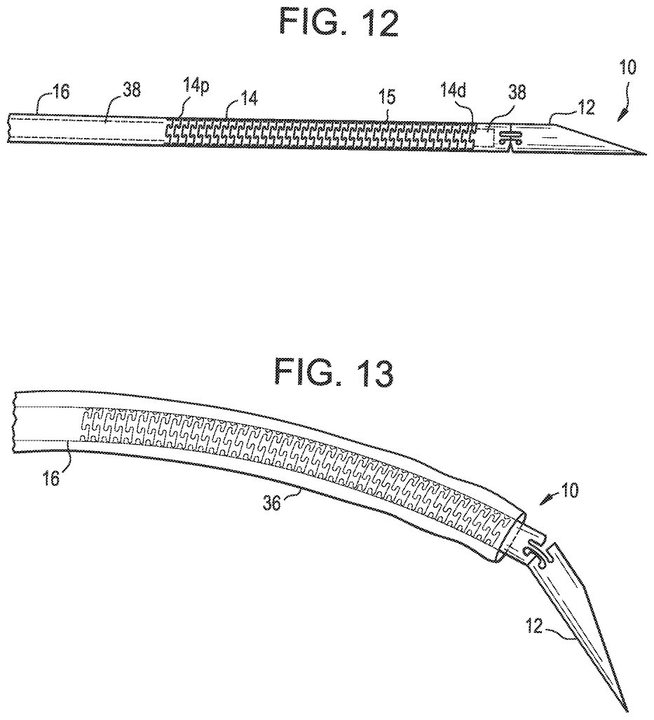

In the illustrated embodiment of FIGS. 8-10, the inner lumen 24 of the needle 10 is either empty or has the flexible elongate tube 32 positioned therein during the movement of the needle 10 from the first configuration to the second configuration and to the third configuration from the second configuration. In another embodiment, as shown in FIG. 12, a mandrel 38 is positioned in the inner lumen 24 of the needle 10 at least when the needle 10 is in the first configuration. The mandrel 38 is a rigid elongate member configured to provide additional structural support to the needle 10. In an exemplary embodiment, the mandrel 38 is non-cannulated. The mandrel 38 allows the hollow needle 10 to effectively act as a solid needle when the mandrel 38 is present therein. The mandrel 38 is located proximal to the hinge 18, and thus not located in the distal tip 12, during the needle's movement from the first configuration to the second configuration. The mandrel 38 is either not located in the intermediate portion 10i or is located in only a proximal portion thereof during the needle's movement from the second configuration to the third configuration. In embodiments in which the flexible elongate tube 32 or other surgical instrument is introduced into the needle 10, the mandrel 38 is removed from the inner lumen 24 prior to the introduction of the flexible elongate tube 32 or other surgical instrument into the inner lumen 24. In FIG. 12, the mandrel 38 is positioned along the entire length of the cut 15 (or, for embodiments including discrete segments instead of the cut 15, within all of the segments), which may prevent the intermediate portion 10i from bending. Proximal retraction of the mandrel 38 from within the intermediate portion 10i allows the intermediate portion 10i to bend.

In another embodiment, instead of the medication 34 and/or other surgical instrument being delivered through the flexible elongate tube 32 positioned in the inner lumen 24 of the needle 10, the medication 34 and/or the surgical instrument can be delivered through a flexible sheath 36 advanced over the needle 10, as shown in FIG. 13. Before delivery of the medication 34 and/or the advancement of the surgical instrument through the sheath 36, the needle 10 is moved in a proximal direction D2 to be removed from an inner lumen of the sheath 36. The medication 34 and/or the surgical instrument may then be passed through the inner lumen of the sheath 36. The needle 10 thus acts as a guide for the sheath 36 to be positioned in a desired position relative to the soft tissue T for delivery of the medication 34 and/or the surgical instrument thereto.

In another embodiment, instead of being advanced over the needle 10 after the needle 10 has been moved to its third configuration, the needle 10 can be at least partially disposed in the inner lumen of the sheath 36, e.g., with the proximal and intermediate portions 10p, 10i in the sheath's inner lumen, when the needle 10 is in the first configuration, when the needle 10 moves from the first configuration to the second configuration, and when the needle 10 moves from the second configuration to the third configuration.

All of the devices and systems disclosed herein can be designed to be disposed of after a single use, or they can be designed to be used multiple times. In either case, however, the devices can be reconditioned for reuse after at least one use. Reconditioning can include any combination of the steps of disassembly of the devices, followed by cleaning or replacement of particular pieces, and subsequent reassembly. In particular, the devices can be disassembled, and any number of the particular pieces or parts of the device can be selectively replaced or removed in any combination. Upon cleaning and/or replacement of particular parts, the devices can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

It can be preferred that devices disclosed herein be sterilized before use. This can be done by any number of ways known to those skilled in the art including beta or gamma radiation, ethylene oxide, steam, and a liquid bath (e.g., cold soak). An exemplary embodiment of sterilizing a device is described in more detail in U.S. Pat. No. 8,114,345 issued Feb. 14, 2012 and entitled “System And Method Of Sterilizing An Implantable Medical Device.”

The present disclosure has been described above by way of example only within the context of the overall disclosure provided herein. It will be appreciated that modifications within the spirit and scope of the claims may be made without departing from the overall scope of the present disclosure.

1-13. (canceled)

14. A medical needle, comprising:
a distal portion including a pointed tip and having an asymmetrical shape, the distal portion being configured to be advanced into a soft tissue;
a proximal portion; and
an intermediate portion located between the distal and proximal portions, the intermediate portion including a plurality of discrete segments each including a plurality of male members on one of a proximal end and distal end thereof and a plurality of female members on the other of the proximal end and distal end thereof, adjacent ones of the segments being connected to one another only by interconnection of the male members and female members;
wherein the pointed tip is configured to bend relative to the intermediate and proximal portions, and the intermediate portion is configured to bend along the segments relative to the proximal portion.

15. The needle of claim 14, wherein the pointed tip is configured to bend at a hinge.

16. The needle of claim 15, wherein the pointed tip is configured to automatically bend at the hinge in response to being advanced in a distal direction through soft tissue.

17. The needle of claim 15, wherein the hinge is a uni-directional hinge that allows the pointed tip to bend in only one direction relative to the intermediate and proximal portions.

18. The needle of claim 17, wherein the pointed tip is defined by a chamfered edge on one side of the distal portion, and the one direction is in a direction opposite to the side of the distal portion having the chamfered edge thereon.

19. The needle of claim 14, wherein the male and female members each have a trapezoidal shape.

20. The needle of claim 14, wherein the segments each have a circular shape with a continuous surface defining an outer perimeter of the circular shape.

21. The needle of claim 14, wherein the segments define a longitudinal member, and the intermediate portion bending along the segments relative to the proximal portion includes the longitudinal member moving from a first configuration, in which longitudinal axes of all of the segments are coaxial with one another, to a second configuration, in which the longitudinal axes of at least some of the segments are not coaxial with one another.

22. The needle of claim 14, wherein the distal, proximal, and intermediate portions are cannulated.

23. The needle of claim 14, wherein the distal, proximal, and intermediate portions are not cannulated.

24. The needle of claim 14, wherein the distal portion is not cannulated, and the intermediate portion is cannulated.

25. The needle of claim 14, wherein the distal, proximal, and intermediate portions are metal.

26-48. (canceled)
